(19) United States
(12) Patent Application Publication
(10) Pub. No.: US 2008/0115785 A1
(43) Pub. Date: May 22, 2008

(54) Inhalers

(75) Inventors: Stephen William Eason, Norfolk (GB); Quentin John Harmer, Cambridge (GB); Matthew Neil Sarkar, Cambridge (GB); John Pinon, Suffolk (GB); Michael John Dunkley, Cambridge (GB); Roger William Clarke, Cambridgeshire (GB); Stephen Henry Hill, Wotton-U-Edge (GB)

Correspondence Address: Davidson, Davidson & Kappel, LLC, 485 7th Avenue, 14th Floor, New York, NY, 10018, US

(73) Assignee: Vectura Delivery Devices Limited, Wiltshire (GB)

(21) Appl. No.: 11/980,929

(22) Filed: Oct. 31, 2007

Related U.S. Application Data
(62) Division of application No. 10/477,055, filed on Jun. 17, 2004.

(30) Foreign Application Priority Data
May 10, 2001 (GB) 0111461.0
Jul. 13, 2001 (GB) 0117138.8
Oct. 12, 2001 (GB) 0124590.1

(51) Int. Cl. A61M 15/00

(52) U.S. Cl. 128/203.15

(57) ABSTRACT

An inhaler for producing an inhalable aerosol of a powdered medicament includes an aerosolizing device in the form of a vortex chamber 1. The vortex chamber 1 has a tangential inlet port 3 and an axial port 2. The ratio of the diameter of the vortex chamber 1 to the diameter of the exit port 2 is between 4 and 12. The length of the exit port 2 is less than its diameter. The cross-section of the inlet port 3 is rectangular and is defined at the bottom and at the radially outermost edge by the walls of the vortex chamber 1. The cross-sectional area of the inlet conduit 7, which supplies the medicament in a gas flow to the inlet port 3, decreases in the direction towards the vortex chamber 1. The inlet conduit 7 may be curved. The inhaler is capable of repeatably producing an aerosol of a medicament with a high proportion of particles in the range 1 to 3 microns, while using a 20 relatively small amount of energy.

### BACKGROUND OF THE INVENTION

The present invention relates to inhalers and in particular inhalers for the delivery of a medicament to the lung, more particularly a medicament in powder form.

In recent times, there has been a growing interest in the systemic delivery of pharmaceutically-active medicaments via the lung. Such a method of delivery is generally more attractive to the patient than methods such as injection, because it does not involve a needle and can be carried out discreetly in public.

For a medicament in a particulate form the provision of an inhalable aerosol requires an inhaler that can produce a repeatable dose of fine particles. In order for the particles of medicament to reach the lung and thus be absorbed into the bloodstream, the particles must have an effective diameter in the range of approximately 1 to 3 microns. The portion of the emitted aerosol within this range of particle size is known as the “fine particle fraction”. If the particles are larger than 5 microns they may not be transported by the inhaled airflow deep into the lung, because they are likely to be trapped in the respiratory passages before reaching the deep lung. For example, particles of the order of 10 microns are unlikely to progress further than the trachea and particles of the order of 50 microns tend to deposit on the back of the throat when inhaled. Furthermore, if the particles are less than 1 micron in effective diameter, the particles may not be absorbed in the lung, because they are small enough to be expelled from the lung with the exhaled airflow.

Thus, it will be seen that it is important that a powdered medicament is delivered with an accurately controlled range of particle size in order that it is absorbed effectively in the lung.

In traditional metered dose inhalers (MDIs) it is common for the emitted dose (the amount of medicament that enters the patient's airway) to be around 80 to 90% of the dose ejected from the inhaler. The fine particle fraction may be only around 50% of the emitted dose. However, the variation in the fine particle fraction of known inhalers can be ±20 to 30%. Such variation may be acceptable in the case of asthma drugs and the like, but when the medicament is a more potent drug such as insulin, growth hormone or morphine, this amount of variability in the dosing is unacceptable. The relatively low fine particle fraction also represents a significant wastage of what may be an expensive drug. Furthermore, there may be side effects if the proportion of the emitted dose which is not respired is swallowed.

Thus, it is important for the systemic delivery of medicaments by inhalation that a repeatable dose of fine particles can be produced.

WO 90/15635 describes a device for the pulverization of particles or agglomerates of a powdered inhalation medicament comprising a rotationally symmetrical vortex chamber with spaced inlet and outlet ports. The inlet port directs air inflow into the vortex chamber substantially parallel to the tangent of the chamber. In one arrangement the chamber has a central outlet port. According to this document the optimum diameter of a vortex chamber operating by the action of inhalation is 10-20 mm. A cylinder with a diameter of 4 mm is disclosed for use with a source of pressurised air.

WO 01/00262 discloses an inhaler comprising a pump, a drug dosing device and a cyclone, which delivers an aerosol of powdered medicament from the drug dosing device into a chamber when the pump is activated. The aerosol is inhaled by the user through a mouthpiece. The cyclone comprises a cylindrical chamber with an axial outlet and a tangential inlet. The cyclone has a preferred diameter between 4 and 10 mm.

Particles of medicament can be separated by generating shear forces between the particles, for example by providing a substantial velocity gradient across the particles. This may be done, for example, by forcing the powder through a narrow nozzle at high speed or introducing the powder into a turbulent air stream. Alternatively, a cyclone of the type described in WO 01/00262 can be used.

It is known for so-called “spacers” to be used in the generation of the aerosol from a metered dose inhaler. The spacer fits onto the mouthpiece of the inhaler and comprises a chamber into which the dose of medicament is ejected by the inhaler. The patient is then able to inhale the dose from the spacer through a corresponding mouthpiece on the spacer. Such spacers retain a fast-moving aerosol ejected from the inhaler, and hold it until it can be inhaled by the user. However, a proportion of the particles in the aerosol will be retained on the walls of the spacer which makes it difficult to predict reliably the dose of medicament that the user inhales. Furthermore, the larger size of the spacer makes the inhaler more cumbersome and less discreet.

### SUMMARY OF THE INVENTION

The present invention, at least in its preferred embodiments, seeks to provide an inhaler which is capable of reliably generating an inhalable aerosol of a powdered medicament with an effective particle size that is sufficiently small for the medicament to be delivered to and absorbed in the lungs of a patient.

Viewed from a first aspect, the invention provides an inhaler for producing an inhalable aerosol of a powdered medicament comprising an aerosolizing device in the form of a vortex chamber of substantially circular cross-section having a substantially tangential inlet port and a substantially axial exit port, wherein the ratio of the diameter of the vortex chamber to the diameter of the exit port is between 4 and 12.

Thus, according to the invention, the aerosolizing device of the inhaler is arranged such that a flow of gas entering the vortex chamber through the inlet port is guided in a rotating path until it leaves the vortex chamber via the exit port. The exit port is generally aligned with the axis of the rotation of the gas flow. When a powdered medicament is entrained in the gas flow, shear forces due to the velocity gradient in the boundary layer adjacent the wall of the vortex chamber break up the agglomerated particles of medicament to form an aerosol of fine particles.

The inlet port can be considered as the end portion of an inlet conduit through which a gas flow enters the chamber, in use. Similarly, the exit port can be considered as the beginning portion of an exit conduit through which the gas flow exits the vortex chamber, in use. An axial exit port directs the gas flow out of the vortex chamber in a substantially axial direction or with a substantial component in the axial direction.

The inventors have realised that the ratio of the diameter of the vortex chamber to the diameter of the exit port is significant in maximising the fine particle fraction of the medicament aerosol which is expelled from the exit port. It has been found that when the ratio is between 4 and 12 the proportion of particles of the powdered medicament with an effective diameter in the range 1 to 3 microns is maximised. For an enhanced fine particle fraction, the ratio is preferably greater than 5, most preferably greater than 6 and preferably less than 9, most preferably less than 8. In the preferred arrangement, the ratio is 7.1.

In embodiments of the invention, the diameter of the vortex chamber is between 2 and 12 mm. The diameter of the vortex chamber is preferably greater than 4 mm, most preferably at least 5 mm and preferably less than 8 mm, most preferably less than 6 mm. In the preferred embodiment, the diameter of the vortex chamber is 5 mm.

In embodiments of the invention, the height of the vortex chamber is between 1 and 8 mm. The height of the vortex chamber is preferably less than 4 mm, most preferably less than 2 mm. In the preferred embodiment, the height of the vortex chamber is 1.6 mm.

In general, the vortex chamber is substantially cylindrical. However, it is within the scope of the invention for the vortex chamber to take other forms. For example, the vortex chamber may be frustoconical. Where the diameter of the vortex chamber or the exit port is not constant along its length, the ratio of the largest diameter of the vortex chamber to the smallest diameter of the exit port should be within the range according to the invention.

In embodiments of the invention, the diameter of the exit port is between 0.5 and 2.5 mm. The diameter of the exit port is preferably greater than 0.6 mm and preferably less than 1.2 mm, most preferably less than 1.0 mm. In the preferred embodiment, the diameter of the exit port is 0.7 mm.

The exit port may comprise a plurality of apertures or passageways. In this case, the diameter of the exit port is considered as the diameter of the smallest circle which circumscribes all of the apertures or passageways which form the exit port.

The inhaler may comprise an exit conduit through which the medicament aerosol passes after leaving the vortex chamber. The exit port may form part of the exit conduit nearest the vortex chamber. If the exit conduit is short, the exit port may form all of the exit conduit.

The exit conduit may be in the form of a tube. The inventors have found, however, that deposition of the aerosolized medicament can occur in a tubular exit conduit, which leads to uncertainty in the dose emitted by the inhaler. Nevertheless, a long exit conduit decreases the plume angle of the medicament aerosol as it exits the conduit and therefore reduces the deposition on the mouthpiece. However, this may increase deposition in the user's throat. Preferably, therefore, the length of the exit conduit or port is short, for example less than the diameter of the exit port. A short exit conduit (or port) increases the plume angle of the medicament aerosol as it exits the conduit (or port) and therefore decreases the speed of the aerosol to reduce deposition in the user's throat.

This in itself is believed to be a novel feature and thus viewed from a second aspect, the invention provides an inhaler for producing an inhalable aerosol of a powdered medicament comprising an aerosolizing device in the form of a vortex chamber of substantially circular cross-section having a substantially tangential inlet port and an exit port, wherein the length of the exit port is less than the diameter of the exit port. In a preferred arrangement, the length of the exit port is less than half the diameter of the exit port. The exit port may be an axial exit port.

Where the diameter of the exit port is not constant along its length, the length of the portion of the exit port having the smallest diameter should be less than that diameter.

In general, the exit port may be defined as a passage through a wall of the vortex chamber. In this case, the length of the exit port may depend on the thickness of the wall. The wall, or a portion thereof, may be tapered (or otherwise reduced in thickness) towards the exit port so that the length of the exit port is less than the maximum thickness of the wall. In particular, the perimeter of the exit port may be in the form of a knife-edge, i.e. a region of negligible thickness.

The wall in which the exit port is defined may be any wall of the vortex chamber. In a preferred arrangement, the exit port is defined in an upper wall of the vortex chamber. The upper wall may have an inner surface which defines the top surface of the chamber, and the furthest extent of the vortex chamber from the inlet port in the axial direction. The inner surface may have any suitable form. For example, the inner surface may be conical, frustoconical, arcuate or hemispherical. In a preferred arrangement, however, the inner surface is planar. In particular, the inner surface may be substantially perpendicular to the axial direction. It has been found that such a configuration maximises the fine particle fraction of the emitted aerosol. The bottom surface of the chamber may also be planar, and the chamber may include a curved lateral surface to provide the substantially circular cross-section.

In certain embodiments of the present invention, the inhaler comprises a chamber. The chamber may have a top portion, a bottom portion, and a substantially cylindrical center portion. The inlet port to the chamber may be tangential to the center portion and the top portion may include an exit port. The chamber may include a chamber wall defining a radially outer boundary of the vortex chamber and defining a maximum extent of the inlet port in a radially outward direction of the chamber.

The inlet port may include an upper wall segment, a lower wall segment, a first lateral wall segment, and a second lateral wall segment. The first lateral wall segment may intersect the chamber at an acute angle and the second lateral wall segment may define a portion of the cylindrical center portion of the chamber.

A ratio of a diameter of the cylindrical center portion to a diameter of the exit port may be between 4 and 12. The exit port may also (or alternatively) have a length that is less than its diameter. In certain embodiments, the exit port is co-axial with a longitudinal axis of the cylindrical center portion, and the inlet port may be perpendicular to the longitudinal axis of the cylindrical center portion.

The inlet port may have any suitable cross-section. For example, the inlet port may have a substantially circular cross-section.

In a preferred configuration, the inlet port has an outer wall which defines the maximum extent of the inlet port in the radially outward direction of the vortex chamber. The extent of the outer wall in the axial direction of the vortex chamber is substantially equal to the maximum extent of the inlet port in the axial direction of the vortex chamber. The outer wall is substantially parallel with the wall of the vortex chamber.

This in itself is believed to be a novel feature and thus viewed from a third aspect, the invention provides an inhaler for producing an inhalable aerosol of a powdered medicament comprising an aerosolizing device in the form of a vortex chamber of substantially circular cross-section having a substantially tangential inlet port,

wherein the inlet port has an outer wall which defines the maximum extent of the inlet port in the radially outward direction of the vortex chamber,

the extent of the outer wall in the axial direction of the vortex chamber is substantially equal to the maximum extent of the inlet port in the axial direction of the vortex chamber, and

the outer wall is substantially parallel with the wall of the vortex chamber. The vortex chamber may comprise an exit port, preferably an axial exit port. A portion of the outer wall may form a portion of the wall of vortex chamber.

In accordance with this aspect of the invention, the inlet port is configured such that its radially outer wall is parallel to the wall of the vortex chamber along substantially the entire axial length of the inlet. In this way, a gas flow with entrained particles of medicament is able to enter the vortex chamber across the whole inlet port along a line which is parallel to the wall of the vortex chamber. This arrangement assists in maximising the proportion of the entrained particles which enter the boundary layer adjacent the wall of the vortex chamber where the shear forces generated by the vortex are at a maximum. In the boundary layer, the maximised shear forces produce maximum deagglomeration of the particles of medicament.

In a preferred arrangement, the outer wall of the inlet port is provided by the wall of the vortex chamber. In this way, the entrained particles of medicament are able to enter directly the boundary layer of the vortex across the whole inlet port.

The cross-section of the inlet port in accordance with this aspect of the invention may take any suitable form relative to the outer wall. For example, the inlet port may be wedge-shaped or quadrant-shaped. In the preferred arrangement, for reasons of simplicity, the inlet port is rectangular in cross-section.

The inlet port may have a height in the axial direction up to the height of the vortex chamber. In certain preferred embodiments, the inlet port and in particular, the inlet port opening in the curved lateral wall of the chamber, is at least half the height of the curved lateral wall. The height of the inlet port may be greater than 1 mm and preferably less than 2 mm. In the preferred configuration, the height of the inlet port is 1.1 mm.

The width of the inlet port in the radial direction may be less than 1 mm. Preferably the width of the inlet port is greater than 0.2 mm, more preferably greater than 0.4 mm. The width of the inlet port is preferably less than 0.8 mm, more preferably less than 0.6 mm. In the preferred configuration, the width of the inlet port is 0.5 mm.

Advantageously, the maximum width of the inlet port is substantially equal to the width of the inlet port at the end furthest in the axial direction from the exit port of the vortex chamber. In this way, the particles of medicament entering the vortex chamber through the inlet port are encouraged initially towards the region of the chamber furthest from the exit port where the inlet port is widest. Thus, the residence time of the particles in the vortex chamber is maximised, thereby allowing more time for effective deagglomeration. The width of the inlet port may be constant along its axial extent.

The vortex chamber may comprise a bottom surface which defines the furthest extent of the vortex chamber from the exit port in the axial direction. In a preferred arrangement, the bottom surface also defines the furthest axial extent of the inlet port. According to this arrangement, the bottom wall of the inlet port is provided by the bottom surface of the vortex chamber. It has been found that such a configuration significantly reduces the deposition of medicament in the vortex chamber in use.

This in itself is believed to be a novel feature and thus viewed from a fourth aspect, the invention provides an inhaler for producing an inhalable aerosol of a powdered medicament comprising an aerosolizing device in the form of a vortex chamber of substantially circular cross-section having a substantially tangential inlet port, an exit port spaced from the inlet port in an axial direction, and a bottom surface which defines the furthest extent of the vortex chamber from the exit port in the axial direction, wherein the bottom surface further defines the furthest axial extent of the inlet port from the exit port. The bottom surface need not be flat and, outside of the region of the inlet port, the vortex chamber may extend more or less in the axial direction than the furthest axial extent of the inlet port.

The inhaler may comprise an inlet conduit arranged to supply a gas flow to the inlet port, in use. The gas flow may contain particles of entrained medicament.

The inlet conduit may have a constant cross-sectional area in the tangential direction towards the vortex chamber. Preferably, however, the cross-sectional area of the inlet conduit decreases towards the vortex chamber. Thus, the inlet conduit may taper towards the vortex chamber. In this way, the velocity of a gas flow of constant mass flow rate increases as the flow moves towards the vortex chamber. The increasing velocity reduces the deposition of medicament entrained in the gas flow during its passage through the inlet conduit.

This in itself is believed to be a novel feature and thus viewed from a fifth aspect, the invention provides an inhaler for producing an inhalable aerosol of a powdered medicament comprising an aerosolizing device in the form of a vortex chamber of substantially circular cross-section having a substantially tangential inlet port and an inlet conduit arranged to supply a gas flow to the inlet port, in use, wherein the cross-sectional area of the inlet conduit decreases towards the vortex chamber.

In embodiments of the invention, the rate of decrease of cross-sectional area with distance of the inlet conduit is between 1% and 30% per millimeter. The rate of decrease is preferably greater than 2% per mm, more preferably greater than 3% per mm and preferably less than 20% per mm, more preferably less than 10% per mm. In the preferred embodiment the rate of decrease is 5% per millimeter.

Preferably, the inlet conduit comprises an outer wall which is substantially tangential to the vortex chamber at the inlet port and an inner wall which converges towards the outer wall in the direction towards the vortex chamber. According to this arrangement, the inner wall guides the incoming gas flow towards the outer wall, such that the gas flow is directed towards the boundary layer of the vortex inside the vortex chamber.

The inlet conduit may be straight, for example the outer wall and the inner wall may be rectilinear. It is within the scope of the invention that only one of the outer wall and the inner wall is rectilinear. In an advantageous embodiment, the inlet conduit is arcuate. This has the advantage that angular momentum is imparted to the incoming gas flow and entrained medicament particles as they pass through the inlet conduit even before they enter the vortex chamber. Thus, the inlet conduit is preferably concavely arcuate relative to the axis of the vortex chamber. The inlet conduit may be arcuate about the axis of the vortex chamber. In this way, the centrifugal force on the incoming gas flow propels the entrained particles of medicament towards the outside edge of the inlet conduit so that the particles enter the vortex chamber adjacent the boundary layer where shear forces are at a maximum.

The curvature of the inlet conduit is preferably sufficient that a tangent to the inner wall at the entrance of the conduit intercepts the outer wall before the end of the conduit. In this way, it is ensured that any particle following a straight path will reach the outer wall of the inlet conduit before entering the vortex chamber.

It is believed that the above arrangement in itself is a novel feature and thus viewed from a sixth aspect the invention provides an inhaler for producing an inhalable aerosol of a powdered medicament comprising an aerosolizing device in the form of a vortex chamber of substantially circular cross-section having a substantially tangential inlet port and an arcuate inlet conduit arranged to supply a medicament entrained in a gas flow to the inlet port, in use.

The arcuate inlet conduit may be any suitable length and have any suitable radius or radii of curvature. In one arrangement, the inlet conduit is in the form of a spiral around the vortex chamber. This arrangement allows a long inlet conduit, for example with only a slight taper, to be provided in a relatively compact way.

The present invention also provides a method for producing an inhalable aerosol of a powdered medicament. The method includes: entraining the powdered medicament in a gas flow upstream from an inlet port of a vortex chamber having a substantially circular cross-section; directing the gas flow through the inlet port into the vortex chamber in a tangential direction; directing the gas flow through the vortex chamber so as to aerosolize the medicament; and directing the gas flow out of the vortex chamber in an axial direction through an exit port, wherein a velocity of the gas flow a short distance (e.g., 50 mm to 300 mm) outside the exit port is less than a velocity of the gas flow at the inlet port.

Inside the vortex chamber, high velocity flows are used to de-agglomerate the powdered drug. In contrast, at the exit port, the inhaler preferably produces a low velocity plume of respirable particles. The device and method according to the present invention thereby provides highly efficient aerosolization using minimal energy and low pressures.

According to a preferred embodiment of this method, at least 80% of the entrained powdered medicament passes through the exit port within 500 ms after the gas flow is directed into the inlet port.

In accordance with another embodiment of the present invention, a method for producing an inhalable aerosol of a powdered medicament is provided. The method comprises entraining a powdered medicament including agglomerated particles in a gas flow upstream from an inlet port of a vortex chamber; directing the gas flow through the inlet port into the vortex chamber; depositing the agglomerated particles onto one or more walls of the vortex chamber; applying, via the gas flow through the vortex chamber, a shear to the deposited agglomerated particles to deagglomerate said particles; and directing the gas flow, including the deagglomerated particles, out of the vortex chamber, wherein a velocity of the gas flow a short distance (e.g., 50 mm to 300 mm) outside the exit port is less than a velocity of the gas flow at the inlet port. In this regard, the powdered medicament may include both agglomerated and unagglomerated particles.

In accordance with other embodiments of the present invention, a method for producing an inhalable aerosol of a powered medicament is provided which comprises: entraining the powdered medicament including agglomerated particles in a gas flow; depositing the agglomerated particles onto one or more surfaces; applying, via the gas flow, a shear to the deposited agglomerated particles to deagglomerate said particles.

In accordance with yet another embodiment of the present invention, a method for producing an inhalable aerosol of a powered medicament is provided which comprises: entraining a powdered medicament including agglomerated particles in a gas flow upstream from an inlet port of a vortex chamber, directing the gas flow through the inlet port into the vortex chamber; depositing the agglomerated particles onto one or more walls of the vortex chamber; applying, via the gas flow through the vortex chamber, a shear to the deposited agglomerated particles to deagglomerate said particles; and directing the gas flow, including the deagglomerated particles, out of the vortex chamber.

In any event, the air flow to the inlet port of the vortex chamber may be generated by the user inhaling and drawing air through the exit port. However, this is not preferred, because the flow rate through the vortex chamber is then dependent on the inhalation rate of the user. It has been found that the fine particle fraction of the medicament aerosol can depend on the flow rate through the vortex chamber.

Thus, in preferred embodiments of the invention, the air flow to the vortex chamber is provided by a source of pressurised air. In this way, an air flow of repeatable volume and velocity can be provided to the vortex chamber in order to minimise variations in the composition of the generated aerosol.

For example, the inhaler may be arranged for connection to a compressed air line or other source of pressurised gas. However, this is not preferred as it is desirable for the inhaler to be self-contained. Consequently, the inhaler may comprise a canister of pressurised gas. The canister may comprise a valve for selectively supplying a gas flow to the vortex chamber. The canister may be rechargeable, for example by means of a pump.

Alternatively, the inhaler may comprise a pump for providing an air flow to the vortex chamber. A pump has the advantage that it does not require recharging or replacing in the manner of a gas canister. The pump may be in any suitable form, for example a squeeze bulb, a bellows pump or such like. A preferred type of pump is a piston pump, in particular a spring-powered piston pump. The piston pump may comprise a plunger received in a pump cylinder. The plunger may be arranged to be withdrawn from the pump cylinder to a primed position against the restoring force of a spring. The plunger may be released when required such that the spring forces the plunger into the pump cylinder to generate an air flow.

In general, the air flow from the pump, canister or other source of pressurised gas is supplied to the vortex chamber via a drug entrainment device.

Thus, the inhaler may comprise a drug entrainment device which is arranged to entrain the powdered medicament in an air flow to the inlet port of the vortex chamber. The drug entrainment device may comprise a substantially cylindrical entrainment chamber having a substantially tangential inlet. The entrainment chamber may also comprise a substantially tangential outlet spaced axially from the inlet.

The inhaler may comprise a mouthpiece and the vortex chamber may be arranged to expel the medicament aerosol into the mouthpiece through the exit port. A mouthpiece locates the vortex chamber relative to the user's airway and allows the medicament aerosol to be directed into the airway. Preferably, the inhaler comprises at least one air passage which allows air to be inhaled through the mouthpiece in addition to the medicament aerosol. The provision of such an air passage allows the user to take a full breath even when the volume of the aerosol is relatively small. The additional air breathed in by a user may be beneficial in propelling the aerosol into the user's lungs.

The inhaler may comprise a breath-actuation device which is arranged to actuate the pump, canister or other source of pressurised gas when the user inhales. The mouthpiece may comprise the breath-actuation device.

In certain preferred embodiments of the present invention, the exit ports described above are located on a top wall of the vortex chamber at a distance R from the vortex chamber axis, wherein R<=⅕ X (more preferably 1/10× or 1/20X), and wherein X is the radius of the vortex chamber. In accordance with further aspects of this embodiment, the exit port may extend through the top wall of the chamber at an angle theta to the axis wherein theta is less than 45 degrees. In this regard, it should be noted that the angle theta is defined with respect to the top wall of the chamber. As such, downstream of the upper wall the direction of the plume may be further altered with a deflector or angled exit tube.

Moreover, in certain embodiments of the present invention, the inlet port described above is substantially tangential to the curved lateral surface of the vortex chamber, and at an angle phi from the normal to the vortex axis wherein the angle phi is in the range +1-45 degrees.

Finally, in certain embodiments of the present invention, the inlet port described above intersects the curved lateral surface of the vortex chamber at an angle beta to true tangent (e.g., measured from the axis of the inlet to true tangent), wherein the angle beta is in the range +/−20 degrees, desirably in the range +/−10 degrees, most desirably in the range +/−5 degrees. This angle beta thereby defines how far the inlet port deviates from being a true tangent to the vortex chamber (when looking from above the chamber).

The terms “axial”, “radial” and “tangential” are used herein to define the geometry of the vortex chamber. These terms are best understood by reference to the vortex formed within the vortex chamber in use. Thus, the axial direction is a direction parallel to the axis about which the vortex rotates. The radial direction is a direction outward from the axis about which the vortex rotates. The tangential direction is a direction parallel to the instantaneous direction of motion of a particle in the vortex. Consequently, it is not necessary for the vortex chamber to have a perfectly circular cross-section, and the vortex chamber need only be sufficiently circular to form an effective vortex. It is desirable for the perimeter of the vortex chamber to form a smooth curve, as it has been found that an angular perimeter can lead to deposition of the medicament in the vortex chamber. It should be noted that the terms top, bottom, and lateral, as used herein, are merely meant to provide reference coordinates, and not to imply a particular orientation when the inhaler is in use.

### BRIEF DESCRIPTION OF THE DRAWINGS

Some embodiments of the invention will now be described by way of example only and with reference to the accompanying drawings, in which:

FIG. 1 is a schematic view, partially in section, of an inhaler according to an embodiment of the invention;

FIG. 2 is a sectional view along line A-A of a detail of the embodiment of FIG. 1;

FIG. 3 is a sectional view, along line C-C of FIG. 4, of a vortex chamber in accordance with the invention;

FIG. 4 is a sectional view along line B-B of the vortex chamber of FIG. 3;

FIG. 5 is a graph of the variation in the fine particle fraction of the aerosol produced by the inhaler of FIG. 1 with variation in the ratio of the diameter of the vortex chamber to that of the exit port;

FIG. 6a is a side view of a vortex chamber with a round inlet port;

FIG. 6b is a sectional view along line D-D of the vortex chamber of FIG. 6a;

FIG. 7a is a side view of a vortex chamber with a rectangular inlet port;

FIG. 7b is a sectional view along line E-E of the vortex chamber of FIG. 7a;

FIG. 8 is a graph of the variation in the fine particle fraction of the aerosol produced by the vortex chambers of FIGS. 6 and 7;

FIGS. 9 to 12 show detail of embodiments of the exit port of the inhaler in accordance with the invention; and

FIG. 13 shows a vortex chamber with an arcuate inlet conduit.

FIG. 14 shows a cutaway view of a vortex camber according to an embodiment of the present invention and approximate air velocities at various points for a flow rate of 3 slpm.

FIG. 15 shows a series of photographs of powder movement through the vortex chamber of FIG. 14.

FIGS. 16a and 16b show schematic views of forces acting on a particle and an agglomeration of particles at a boundary layer of the flow in a chamber.

FIG. 17 shows an example of flow velocities inside the vortex chamber at a cross-section through the axis of the chamber.

FIG. 18 shows a flow rate profile at an inlet to a dose storage device during a dose delivery.

In the various embodiments of the invention, corresponding components are given corresponding reference numerals.

### DETAILED DESCRIPTION OF THE PREFERRED EMBODIMENTS

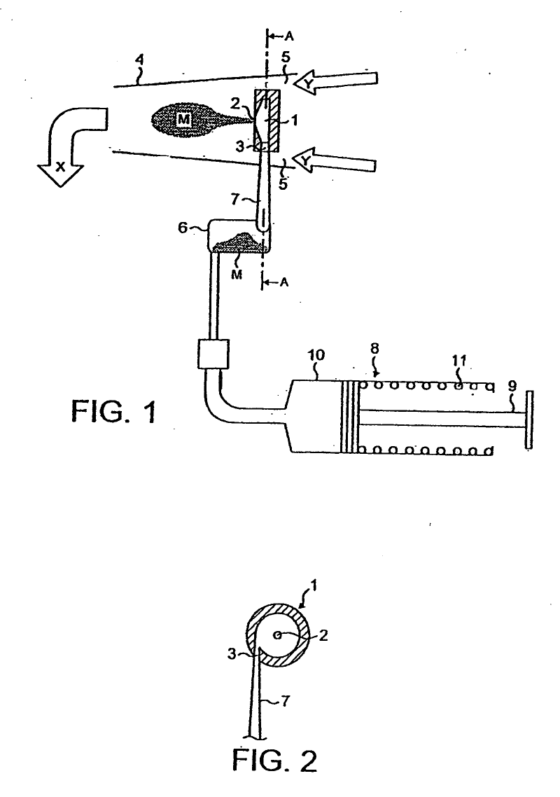

FIG. 1 shows schematically a prototype inhaler according to an embodiment of the invention. The inhaler aerosolizes a drug in dry powder form for inhalation by the user.

As shown in FIG. 1, the inhaler comprises a vortex chamber (or nozzle) 1 having an exit port 2 and an inlet port 3 for generating an aerosol of medicament M. The vortex chamber 1 is located in a mouthpiece 4 through which the user inhales in use of the inhaler, as indicated by the arrow X. Air passages 5 are defined between the vortex chamber 1 and the mouthpiece 4 so that the user is able to inhale air in addition to the medicament aerosol M, as indicated by arrows Y.

The powdered medicament (or drug) M is provided to the vortex chamber 1 in an air flow from a drug entrainment device 6 via an inlet conduit 7. The drug entrainment device 6 is in the form of a cylindrical chamber with tangential inlet and outlet ports spaced in the axial direction. The drug may be supplied for transfer to the drug entrainment chamber in a foil blister or a standard gelatin capsule, containing 1 to 5 milligrams of powdered drug. The optimum particle size of the drug for delivery to the deep lung is 1 to 3 microns. If necessary an inert excipient, such as lactose, can be added to the drug to increase its bulk and improve its handling properties. Non-limiting examples of formulations with which the inhaler may be used are micronised pure drugs such as sodium cromoglicate, terbutaline sulphate and pure salbutamol sulphate, and spray-dried formulations of drugs such as insulin and paracetamol with a carrier such as hydroxy-ethyl starch.

The air flow to the drug entrainment device 6 is provided by a pump 8, represented in FIG. 1 as a spring-powered piston pump. The pump 8 comprises a plunger 9 received in a pump cylinder 10 and biased into the pump cylinder 10 by a spring 11. The pump 8 is selected to have a capacity of less than 100 ml, preferably less than 50 ml and more preferably between 5 and 25 ml in order that the total size of the inhaler is relatively small. The pump B is capable of generating a pressure between 0.5 and 10 bar gauge, preferably less than 5 bar and more preferably less than 2 bar in order that the total size of the inhaler is relatively small. The flow rate through the inhaler is typically 1 to 5 litres per minute and may be adjusted for optimum performance with a particular medicament.

In use of the inhaler, the pump 8 is primed by retracting the plunger 9 against the force of the spring 11. The plunger 9 is retained in the primed position by a breath-actuated mechanism (not shown) until the user inhales. When the user inhales, the plunger 9 is released by the breath-actuated mechanism and the spring 11 forces the plunger 9 in to the pump cylinder 10, which forms a pressurized air reservoir. In this way, air is forced from the pressurized air reservoir through the drug entrainment device 6 where the powdered medicament M is entrained in the air flow. The air flow transports the medicament M to the vortex chamber 1, where a rotating vortex of medicament and air is created between the inlet port 3 and the outlet port 2. Rather than passing through the vortex chamber in a continuous manner, the powdered medicament entrained in the airflow enters the vortex chamber in a very short time (less than 0.3 seconds) and a proportion of the powdered medicament sticks to the walls of the vortex chamber. This powder is subsequently aerosolized by the high shear forces present in the boundary layer adjacent to the powder. The action of the vortex deagglomerates the particles of medicament M so that an aerosol M of powdered medicament exits the vortex chamber 1 via the exit port 2. The aerosol is inhaled by the user through the mouthpiece 4.

The vortex chamber 1 can be considered to perform two functions: deagglomeration, the breaking up of clusters of particles into individual, respirable particles; and filtration, preferentially allowing particles below a certain size to escape more easily from the exit port 2. Deagglomeration breaks up cohesive clusters of powdered medicament into respirable particles, and filtration increases the residence time of the clusters in the vortex chamber 1 to allow more time for them to be deagglomerated. Deagglomeration can be achieved by creating high shear forces due to velocity gradients in the airflow in the vortex chamber 1. The velocity gradients are highest in the boundary layer close to the walls of the vortex chamber.

As shown in more detail in FIG. 2, the vortex chamber 1 is in the form of a substantially cylindrical chamber. The vortex chamber 1 has a frustoconical portion in the region of the exit port 2. The inlet port 3 is substantially tangential to the perimeter of the vortex chamber 1 and the exit port 2 is generally concentric with the axis of the vortex chamber 1. Thus, gas enters the vortex chamber 1 tangentially via the inlet port 3 and exits axially via the exit port 2. Between the inlet port 3 and the exit port 2 a vortex is created in which shear forces are generated to deagglomerate the particles of medicament. The length of the exit port 2 is as short as possible to reduce the possibility of deposition of the drug on the walls of the exit port 2. In the embodiment shown, the vortex chamber 1 is machined from acrylic or brass, although a wide range of alternative materials is possible.

TABLE 1
Vortex chamber dimensions

| | |
|---|---|
| Dimension | Value |

| | | |
|---|---|---|
| D | Diameter of chamber | 5.0 mm |
| H | Height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 12° |

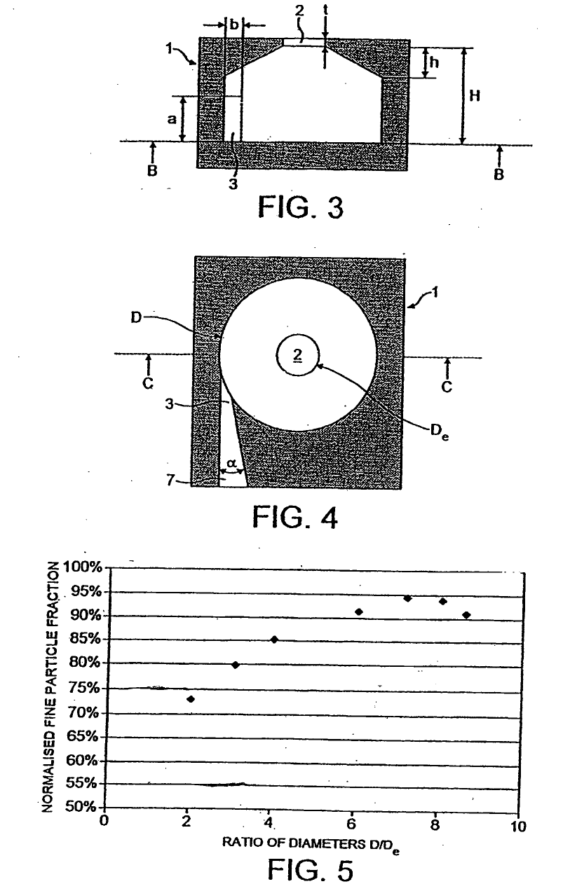

FIGS. 3 and 4 show the general form of the vortex chamber of the inhaler of FIG. 1. The geometry of the vortex chamber is defined by the dimensions listed in Table 1. The preferred values of these dimension are also listed in Table 1. It should be noted that the preferred value of the height h of the conical part of the chamber is 0 mm, because it has been found that the vortex chamber functions most effectively when the top of the chamber is flat.

As shown in FIG. 5, the fine particle fraction of the aerosol generated by the vortex chamber depends on the ratio of the diameters of the chamber D and the exit port Dₒ. The data represented in FIG. 5 is shown in Table 2. The fine particle fraction is the proportion of the particles of medicament emitted in the aerosol having an effective particle diameter of less than 6.8 microns. The normalized fine particle fraction is the emitted fine particle fraction divided by the fine particle fraction of the powdered medicament loaded into the inhaler. The medicament used was pure sodium cromoglicate.

TABLE 2
Relationship between emitted fine particle fraction and
ratio of vortex chamber diameter to exit port diameter.

| Ratio | Average fine particle | Normalised average fine |
|---|---|---|
| D/Dₑ | fraction (<6.8 um) | particle fraction |
| 2.0 | 64.7% | 73.1% |
| 3.1 | 70.8% | 79.9% |
| 4.0 | 75.5% | 85.2% |
| 6.0 | 81.0% | 91.4% |
| 7.1 | 83.5% | 94.3% |
| 8.0 | 83.2% | 93.9% |
| 8.6 | 80.6% | 91.0% |

It will be seen from FIG. 5 that where the ratio of the diameters of the chamber and the exit port is 4 or more, the normalized fine particle fraction is over 85%. Thus, the deagglomeration efficiency of the vortex chamber is significantly improved where the ratio is in this range. With the preferred ratio of 7.1, a normalized fine particle fraction of 94.3% has been achieved.

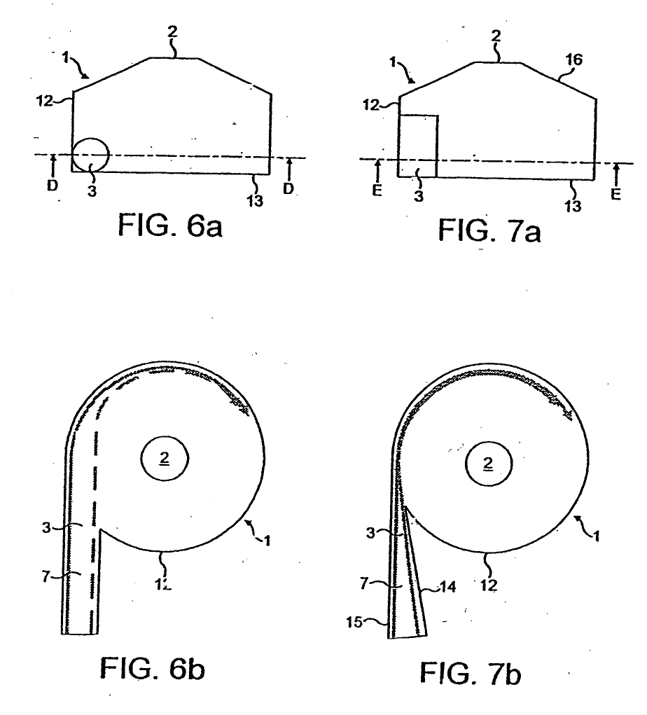

FIGS. 6a and 6b show a vortex chamber 1 in which the inlet port 3 has a circular cross-section. As represented by the solid arrow in FIG. 6b, a proportion of the airflow entering the vortex chamber via the inlet port 3 follows the lateral wall 12 of the vortex chamber 1. The medicament entrained in this airflow is therefore introduced directly into the airflow at the boundary layer adjacent the lateral wall 12 of the vortex chamber 1, where the velocity gradient in the radial direction is at a maximum. The maximal velocity gradient results in maximal shear forces on the agglomerated particles of medicament and thus maximum deagglomeration.

However, as represented by the dashed arrow in FIG. 6b, a proportion of the airflow entering the vortex chamber via the inlet port 3 does not follow the chamber wall 12, but rather crosses the chamber 1 and meets the wall 12 at a point opposite the inlet port 3. At this point, there is increased turbulence, because the flow must make an abrupt change of direction. This turbulence disturbs the boundary layer adjacent the wall 12 of the chamber 1 and thereby reduces the effectiveness of the deagglomeration of the medicament.

FIGS. 7a and 7b show a vortex chamber 1 in which the inlet port 3 has a rectangular cross-section. The rectangular cross-section maximises the length of the perimeter of the inlet port that is coincident with the wall 12 of the vortex chamber 1, such that the maximum air flow is introduced into the boundary layer of the vortex. Similarly, the rectangular cross-section maximises the width of the perimeter of the inlet port 3 that is coincident with the bottom surface 13 of the vortex chamber 1. In this way, deposition of medicament in the vortex chamber 1 is prevented, because the vortex occupies the entire chamber 1.

In addition to having a rectangular cross-section, the inlet port 3 of FIGS. 7a and 7b is supplied by an inlet conduit 7 which tapers towards the vortex chamber 1. Thus, the inlet conduit 7 is defined by an inner wall 14 and an outer wall 15. The outer wall 15 is substantially tangential to the wall 12 of the vortex chamber 1. The spacing of the inner wall 14 from the outer wall 15 decreases towards the vortex chamber 1, so that the inner wall 14 urges the air flow into the vortex chamber 1 towards the boundary layer. Furthermore, the decreasing cross-sectional area of the inlet conduit 7 causes the flow of velocity to increase, thereby reducing deposition of medicament on the way to the vortex chamber 1.

As indicated by the arrows in FIG. 7b, all of the airflow entering the vortex chamber via the inlet port 3 follows the wall 12 of the vortex chamber 1. The medicament entrained in this airflow is therefore introduced directly into the airflow at the boundary layer adjacent the wall 12 of the vortex chamber 1, and deagglomeration is maximised.

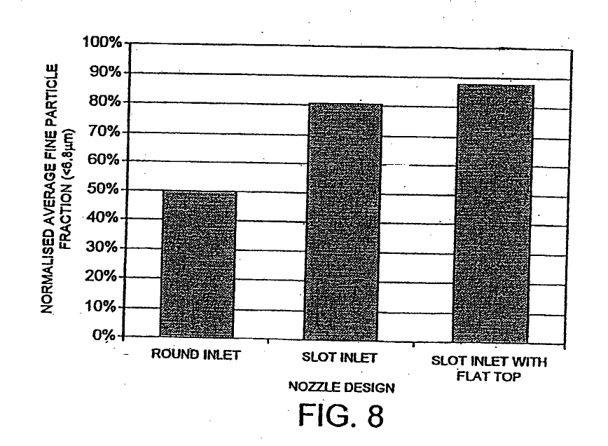

FIG. 8 shows that the average normalized fine particle fraction produced by the vortex chamber 1 of FIGS. 6a and 6b is only 49.7% compared to an average normalized fine particle fraction of 80.3% for the vortex chamber 1 of FIGS. 7a and 7b having an inlet port in the form of a slot of rectangular cross-section.

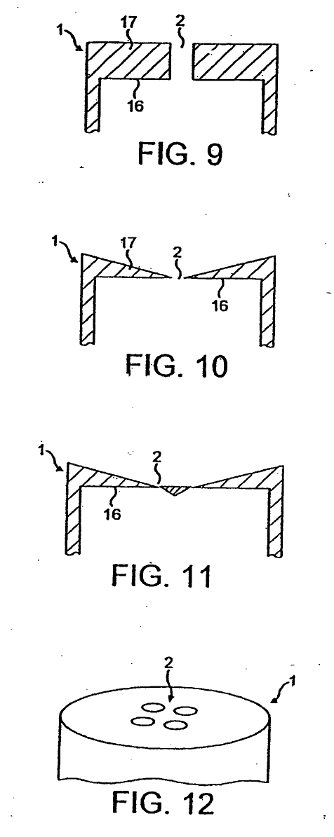

A further improvement can also be achieved if the upper surface 16 of the vortex chamber 1 is flat, as shown in FIGS. 9 to 11, rather than conical as shown in FIGS. 1, 3, 6 and 7. Thus, in this arrangement, the upper surface 16 of the vortex chamber 1 is substantially perpendicular to the wall 12 of the chamber 1, and to the axis of the vortex. As shown in FIG. 8, the average normalized fine particle fraction produced by the vortex chamber 1 with a flat upper surface (or top) is 87.8% compared to an average normalized fine particle fraction of 80.3% where the upper surface is conical.

FIGS. 9 to 12 show various options for the exit port 2 of the vortex chamber 1. The characteristics of the exit plume of the aerosol are determined, at least in part, by the configuration of the exit port 2. For example, if the aerosol leaves an exit port 2 of 1 mm diameter at a flow rate of 2 litres/minute, the velocity at the exit port 2 will be approximately 40 m/s. This velocity can be reduced to a typical inhalation velocity of 2 m/s within a few centimeters of the chamber or nozzle by providing a strongly divergent aerosol plume.

In FIG. 9, the exit port 2 is a simple orifice defined through the upper wall 17 of the vortex chamber 1. However, the thickness of the upper wall 17 means that the exit port 2 has a length which is greater than its diameter. Thus, there is a risk of deposition in the exit port as the aerosol of medicament exits. Furthermore, the tubular exit port tends to reduce the divergence of the exit plume. These problems are solved in the arrangement of FIG. 10 by tapering the upper wall 17 of the vortex chamber 1 towards the exit port 2 so that the exit port 2 is defined by a knife edge of negligible thickness. For an exit port 2 of diameter 1 mm, an exit port length of 2.3 mm gives a plume angle of 60°, whereas reducing this length to 0.3 mm increases the angle to 90°.

In FIG. 11, the exit port 11 is annular and is also defined by a knife edge. This arrangement produces an exit plume that slows down more quickly than a circular jet, because the annular exit port has a greater perimeter than a circular port of the same diameter and produces a jet that mixes more effectively with the surrounding static air. In FIG. 12, multiple orifices form the exit port 2 and produce a number of smaller plumes which break up and slow down in a shorter distance than a single large plume.

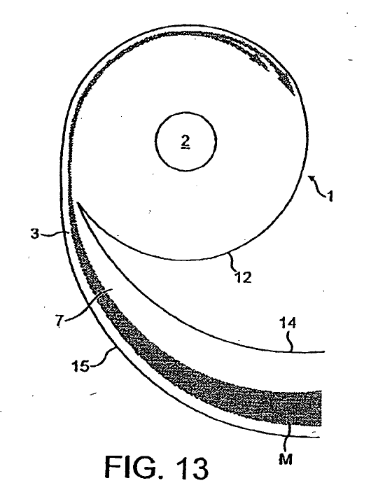

FIG. 13 shows an embodiment of the vortex chamber 1 in which the inlet conduit 7 is arcuate and tapers towards the vortex chamber 1. As shown by the arrows in FIG. 13, the arcuate inlet conduit 7 urges the entrained particles of medicament M towards the outer wall 15 of the inlet conduit 7. In this way, when the medicament enters the vortex chamber 1 through the inlet port 3 the medicament is introduced directly into the boundary layer next to the wall 12 of the vortex chamber 1, where shear forces are at a maximum. In this way, improved deagglomeration is achieved.

TABLE 3
Fine particle fractions (FPF) for topical
delivery of medicament (<6.8 microns)

| | Medicament | Delivered | Deagglomeration |
|---|---|---|---|
| Medicament | FPF | FPF | efficiency |
| Micronised | 88.6% | 83.6% | 94% |
| Sodium | | | |
| Cromoglycate | | | |
| Terbutaline | 85.8% | 70.2% | 83% |
| Sulphate | | | |
| Micronised | 88.7% | 73.7% | 83% |
| Salbutamol | | | |
| Sulphate | | | |

Tables 3 and 4 show the analysis of the aerosol produced by an inhaler according to an embodiment of the invention using an Astra Draco Multi-Stage (⅘) Liquid Impinger (MLI). The performance of the inhaler was tested using three medicament formulations: micronised sodium cromoglicate, terbutaline sulphate and micronised salbutamol sulphate. In each case, the dose of drug was 1 milligram and the flow rate of air through the vortex chamber was 3 litres/minute.

Initially, the fine particle fraction of the powdered medicament before aerosolization was determined, as this represents the maximum achievable fine particle fraction for the aerosol. To determine the initial fine particle fraction, the powdered medicament was fully dispersed in a non-solvent, cyclohexane, by means of ultrasonic agitation and the particle distribution measured using a laser particle sizer available from Malvern Instruments Limited of Malvern UK. For topical delivery of the medicament (Table 3) the fine particle fraction is defined as the proportion of particles with a particle size of less, than 6.8 microns. For systemic delivery of the medicament (Table 4) the fine particle fraction is defined as the proportion of particles with a particle size of less than 3 microns. The fine particle fraction of the aerosol was determined and compared to the corresponding fine particle fraction before aerosolization to give a value for deagglomeration efficiency as a percentage of the maximum achievable fine particle fraction.

TABLE 4
Fine particle fractions (FPF) for systemic
delivery of medicament (<3 microns)

| | Medicament | Delivered | Deagglomeration |
|---|---|---|---|
| Medicament | FPF | FPF | efficiency |
| Micronised | 66.7% | 62.2% | 93% |
| Sodium | | | |
| Cromoglycate | | | |
| Terbutaline | 54.6% | 44.6% | 82% |
| Sulphate | | | |
| Micronised | 57.6% | 52.6% | 91% |
| Salbutamol | | | |
| Sulphate | | | |

The results in Table 3 and 4 show that for each of the three medicaments the deagglomeration efficiency is over 80% for both topical and systemic delivery and in many cases is over 90%.

The inhaler in accordance with embodiments of the invention is able to generate a relatively slow moving aerosol with a high fine particle fraction. The inhaler is capable of providing complete and repeatable aerosolization of a measured dose of powdered drug and of delivering the aerosolized dose into the patient's inspiratory flow at a velocity less than or equal to the velocity of the inspiratory flow, thereby reducing deposition by impaction in the patient's mouth. Furthermore, the efficient aerosolizing system allows for a simple, small and low cost device, because the energy used to create the aerosol is small. The fluid energy required to create the aerosol can be defined as the integral over time of the pressure multiplied by the flow rate. This is typically less than 5 joules and can be as low as 3 joules.

Although the aerosol of medicament has been described herein as an aerosol of powdered medicament in air, the medicament may be dispersed in any other gas or mixture of gases, as required. Furthermore, although the invention has been described in terms of apparatus, the invention also extends to a method of generating an inhalable aerosol of a powdered medicament as described herein.

In summary, an inhaler for producing an inhalable aerosol of a powdered medicament includes an aerosolizing device in the form of a cylindrical vortex chamber. The vortex chamber has a tangential inlet port and an axial exit port. The ratio of the diameter of the vortex chamber to the diameter of the exit port is between 4 and 12. The length of the exit port is less than its diameter. The cross-section of the inlet port is rectangular and is defined at the bottom and at the radially outermost edge by the walls of the vortex chamber. The cross-sectional area of the inlet conduit, which supplies the medicament in a gas flow to the inlet port, decreases in the direction towards the vortex chamber. The inlet conduit can be curved. The inhaler is capable of repeatably producing an aerosol of a medicament with a high proportion of particles in the range 1 to 3 microns, while using a relatively small amount of energy.

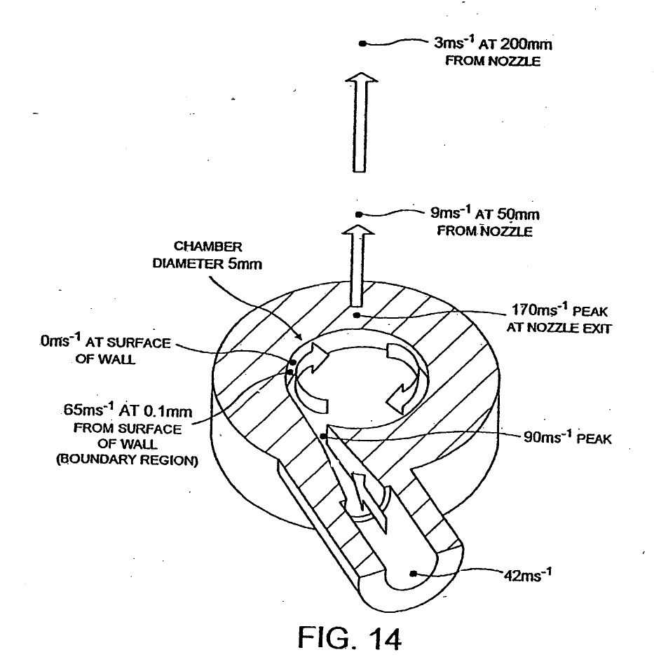

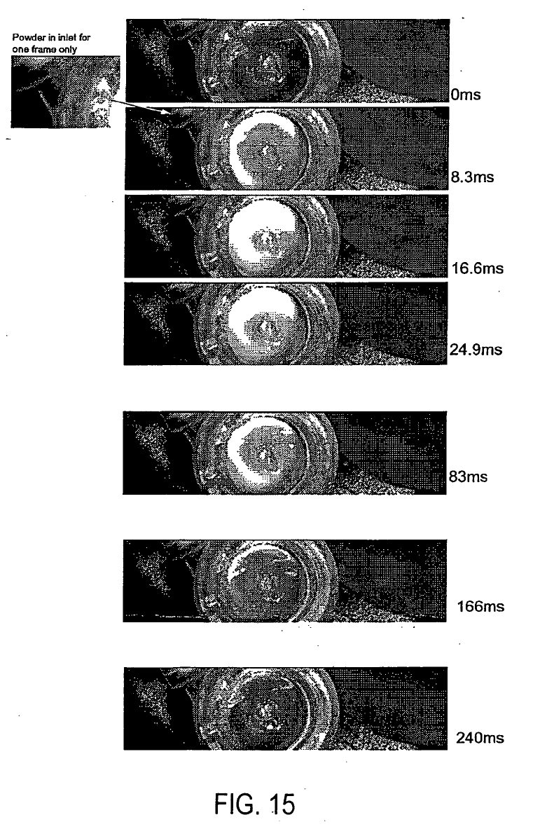

FIG. 14 shows an exemplary vortex chamber in accordance with an embodiment of the present invention, and FIG. 15 shows a series of photographs of powder movement through the device of FIG. 14 during use. The chamber of FIG. 14 is a cylindrical vortex chamber having a chamber diameter of 5 mm, an axial exit port with a diameter of 0.7 mm, and an inlet conduit which tapers towards the chamber terminating in a tangential inlet port which has a rectangular inlet port opening having a width of 0.5 mm and a height of 1.1 mm. As illustrated in FIG. 15, substantially all the powder enters the vortex chamber in less than 8 ms and is smeared around the walls of the chamber. Over the following 250 ms the powder is scoured off the walls and leaves the chamber via the exit port. It is believed that the “stick and scour” behaviour is optimized by the geometry of the vortex chamber, inlet and outlet ports as described herein.

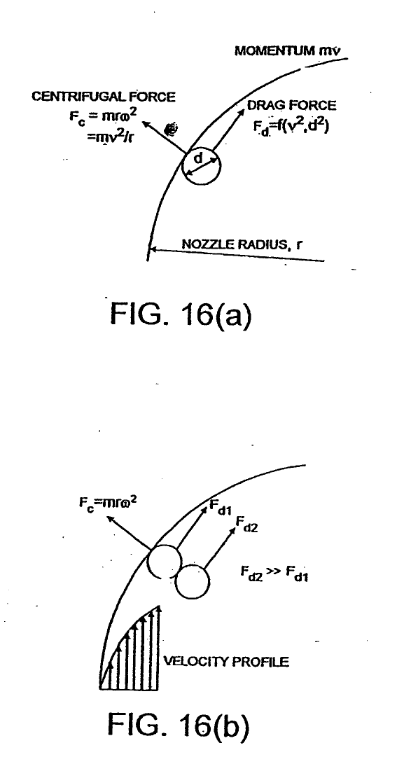

As illustrated in FIGS. 16a, two main forces acting on a particle in the chamber are the centrifugal force tending to move the particle against the curved lateral wall of the vortex chamber and the drag force of the air that carries it along. The powder dose typically enters the chamber over a short period (for example, within 5 ms) and is smeared around the walls of the chamber under the influence of the centrifugal force. The centrifugal acceleration producing this force is many times the acceleration due to gravity. For example, a particle travelling at 65 ms⁻¹ in the chamber will experience an acceleration of 1,690,000 ms⁻² (from a=−v²/r). This is 172,270 times the acceleration due to gravity (g=9.8 ms⁻²) and means that the effect of the centrifugal force is to multiply the weight of a particle by 172,270 times. The aerodynamic drag force acts to carry the particles through the chamber with the airflow and out of the exit. However, only small particles can overcome the centrifugal force and move to the central region of the vortex where they can escape. Larger agglomerates need to be broken up into smaller particles before they can exit the chamber. This deagglomeration occurs close to the chamber wall in the boundary layer.

In the boundary layer, a region close to the wall with a thickness of approximately 0.1 mm, there is a sharp velocity gradient, as shown in the graph of FIG. 15. The velocity gradient (dv/dr) at the boundary layer is in the region of 600 ms⁻¹/mm, an order of magnitude higher than the velocity gradient elsewhere in the chamber.

This sharp gradient means that the drag force acting across the width of an agglomerate or larger particle is not uniform, as can be seen in FIG. 16b. It is believed that this creates a shear across agglomerates of particles that breaks the agglomerates down into their constituent particles. The geometry of the chamber is chosen so that individual particles, once they are separated, are small enough to be drawn to the centre of the vortex under the influence of the drag forces and then exit the chamber. Typically, over a period of around 250 ms, the dose may be aerosolized.

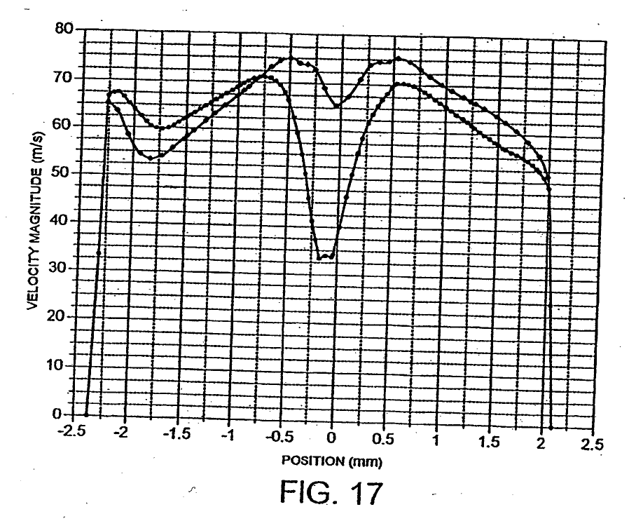

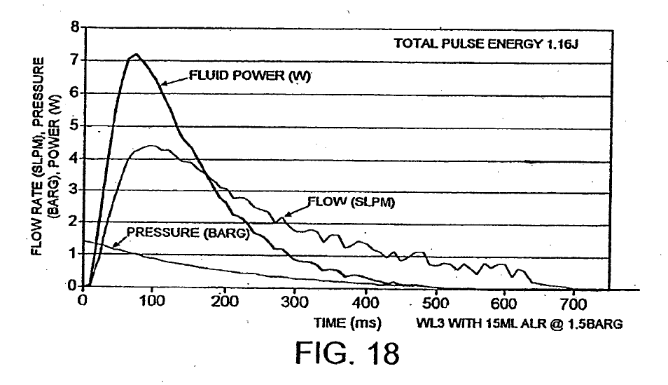

When gas (e.g., air) is released, such as from a pressurized gas reservoir, it flows through a drug entrainment device to entrain the powder (e.g., drug entertainment device 6 of FIG. 1) and then into the vortex chamber where the powder is deagglomerated and exits the chamber as a respirable aerosol. The flow rate through the device varies with time, from zero, to a peak of between 4 and 5 SLPM(standard liters per minute, which is an equivalent flow rate in liters per minute at standard temperature and pressure), but the average value over the time that the powder is delivered is typically between 3 and 4 SLPM as shown in FIG. 17.

Based upon observations of the device of FIGS. 14 and 15, it is believed that, inside the chamber, this flow creates peak velocities of over 150 ms⁻¹. However, the highly swirling flow diffuses rapidly after exiting the chamber, and, by the time the powder is a short distance from the chamber, the plume is travelling at the same velocity as a typical user's inspiratory flow. In this regard, the velocities at various points in the system are shown in FIG. 14 and Table 5. The flow rate at the output port 3 slpm.

TABLE 5
Flow velocities at various points in the device of FIG. 14

| | | Flow | |
|---|---|---|---|
| | Flow cross | velocity at | |
| | section | 3.0 SLPM | |
| | dimensions | flow rate | Source |
| Flow velocity at exit from | Ø1.2 mm | 42 ms⁻¹ | Calculated from |
| dose storage (average) | | | bulk flow rate |
| Flow velocity at inlet to | 0.5 × 1.1 | 90 ms⁻¹ | Calculated from |
| vortex chamber (peak | mm | | CFD model |
| velocity) | | | |
| Plume velocity at exit | Ø0.7 mm | 170 ms⁻¹ | Calculated from |
| plane of vortex chamber | | | CFD model |
| (peak velocity) | | | |
| Plume velocity 50 mm from | Free air | 9 ms⁻¹ | Estimated from |
| chamber exit | | | high speed |
| | | | video |
| Plume velocity 200 mm from | Free air | 3 ms⁻¹ | Estimated from |
| chamber exit | | | high speed |
| | | | video |
| User breathing through | Ø20 mm | 3 ms⁻¹ | Calculated from |
| typical Ø20 mm inhaler | | | bulk flow rate |
| mouthpiece at 60 SLPM | | | |

In the preceding specification, the invention has been described with reference to specific exemplary embodiments thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1-49. (canceled)

50. A method for producing an inhalable aerosol of a powdered medicament, the method comprising:
entraining the powdered medicament in a gas flow upstream from an inlet port of a vortex chamber having a substantially circular cross-section,
directing the gas flow through the inlet port into the vortex chamber in a tangential direction;
directing the gas flow through the vortex chamber so as to aerosolize the medicament; and
directing the gas flow out of the vortex chamber in an axial direction through an exit port, wherein a velocity of the gas flow at a distance of 300 mm outside of the exit port is less than a velocity of the gas flow at the inlet port.

51. The method as recited in claim 50 wherein at least 80% of the entrained powdered medicament passes through the exit port within 500 ms after the gas flow is directed into the inlet port.

52. The method of claim 50, wherein the velocity of the gas flow at a distance of 50 mm outside of the exit port is less than the velocity of the gas flow at the inlet port. 53.

53. The method of claim 50, wherein the gas flow upstream of the inlet port is generated by a source of pressurized gas.

54. A method for producing an inhalable aerosol of a powdered medicament, the method comprising:
entraining a powdered medicament including agglomerated particles in a gas flow upstream from an inlet port of a vortex chamber, directing the gas flow through the inlet port into the vortex chamber;
depositing the agglomerated particles onto one or more walls of the vortex chamber;
applying, via the gas flow through the vortex chamber, a shear to the deposited agglomerated particles to deagglomerate said particles,
directing the gas flow, including the deagglomerated particles, out of the vortex chamber, wherein a velocity of the gas flow at a distance of 300 mm outside of the exit port is less than a velocity of the gas flow at the inlet port.

55. The method of claim 54, wherein the velocity of the gas flow at a distance of 50 mm outside of the exit port is less than the velocity of the gas flow at the inlet port.

56. The method of claim 54, wherein the gas flow upstream of the inlet port is generated by a source of pressured gas.

57. A method for producing an inhalable aerosol of a powered medicament, the method comprising: entraining the powdered medicament including agglomerated particles in a gas flow;
depositing the agglomerated particles onto one or more surfaces;
applying, via the gas flow, a shear to the deposited agglomerated particles to deagglomerate said particles.

58. A method for producing an inhalable aerosol of a powered medicament, the method comprising:
entraining a powdered medicament including agglomerated particles in a gas flow upstream from an inlet port of a vortex chamber,
directing the gas flow through the inlet port into the vortex chamber; depositing the agglomerated particles onto one or more walls of the vortex chamber;
applying, via the gas flow through the vortex chamber, a shear to the deposited agglomerated particles to deagglomerate said particles; and
directing the gas flow, including the deagglomerated particles, out of the vortex chamber.

59-65. (canceled)
